(19) United States
(12) Patent Application Publication
(10) Pub. No.: US 2026/0357172 A1
(43) Pub. Date: Nov. 12, 2026

(54) CELL CULTURE DEVICES

(71) Applicants: VAL-CHUM, LIMITED PARTNERSHIP, Quebec (CA); POLYVALOR, LIMITED PARTNERSHIP, Quebec (CA)

(72) Inventors: Etienne LAURENT, Montreal (CA); Samuel BOURDON, Montreal (CA); Thomas GERVAIS, Montreal (CA); Dina DORRIGIV, Montreal (CA); Rodin CHERMAT, Montreal (CA); Amelie ST-GEORGES-ROBILLARD, Montreal (CA); Elena REFET-MOLLOF, Montreal (CA); Gabriel PAGE, Montreal (CA)

(21) Appl. No.: 19/125,841

(22) PCT Filed: Oct. 31, 2023
(86) PCT No.: PCT/CA2023/051446

§ 371 Date: Apr. 30, 2025

Related U.S. Application Data
(60) Provisional application No. 63/420,834, filed on Oct. 31, 2022.

(51) Int. Cl. C12M 1/32; C12M 1/00; C12M 1/04; C12M 1/12

(52) U.S. Cl. CPC C12M 23/12; C12M 23/22; C12M 23/24; C12M 23/38; C12M 25/14; C12M 29/04; C12M 29/18

(57) ABSTRACT

A cell culture device comprising: a chip component having: a top face and a bottom face, a channel formed in the chip component which is open at the top face and has a base spaced from the bottom face; a well portion extending from the base of the channel to the bottom face of the chip component, the well portion being open at the bottom face of the chip component and at the base of the channel; and a membrane component configured to be positioned on the bottom face of the chip component to close the open well portion, the membrane component having a porous portion which is aligned with the open well portion when the membrane component and the chip component are assembled together, the porous portion of the membrane component being permeable to gas and impermeable to liquid.

### TECHNICAL FIELD

The present technology generally relates to cell culture devices, suitable for, but not limited to, microfluidic cell culture.

### BACKGROUND

Being able to predict a patient's response to various therapeutic modalities, before any treatment is given to the patient, remains a goal of personalized medicine. Such predictive information assists doctors in tailoring an effective treatment plan based on individual characteristics and providing the appropriate treatment to the patient. The requirement of personalized medicine is even more pressing for complex diseases like cancer where many therapeutic modalities exist including combinations of treatments. Predictive information can also contribute to drug development processes in terms of enhancing the preclinical testing phase and providing more certainty about drug candidates before the clinical phase.

The objectives of predicting patient responses and enhancing the preclinical-clinical translation has led to the development of 3D culture model systems for simulating human physiology. These systems are also referred to as complex in vitro systems. Compared to conventional in vitro 2D cell culture techniques, 3D culture models provide a more physiologically realistic simulated environment for culturing and testing living cells in a laboratory context.

Generally, 3D culture models comprise an artificially created environment that permits cells to grow in three dimensions, as compared to a two dimensional environment such as a petri dish. 3D culture models essentially comprise a cell culture device in which is cultured cells. 3D culture models typically comprise scaffold-based methods using hydrogels for example, as well as scaffold-free approaches using freely floating aggregates referred to as spheroids. Compared to 2D culture techniques, 3D culture models typically (i) use, or are derived from, human cells instead of immortalized cell lines or animal cells, (ii) enable the 3D growth of cells, sometimes recreating the cellular architecture of organs, (iii) allow the perfusion of nutrients or biological fluids to the cells, and (iv) reproduce specific physiological functions in order to assess the efficacy or the toxicology of a therapeutic.

Even though existing 3D culture models have certain benefits over conventional 2D in vitro techniques, limitations still remain. To this end, there is an interest in developing improved methods and systems for 3D cell culture.

### SUMMARY

Embodiments of the present technology have been developed based on developers' appreciation of at least one technical problem associated with the prior art solutions.

In some 3D culture models of the prior art, the cells to be cultured (also referred to herein as “cell model”) comprise monoculture or co-culture spheroids. These spheroids are simple three-dimensional cell aggregates usually used in high-throughput systems for drug discovery or even for personalized treatment.

In some 3D culture models, the cell model comprises organoids. The organoids are 3D microtissues grown from isolated cells to recreate the arrangement of a target tissue or organ. The organoids require the use of a substrate such as matrigel™ as an extracellular matrix to support the cells to grow into 3D mini-organs.

However, both these existing cell model types require prior growth of the cells into a 3D architecture. The 3D architecture however lacks the native tumor microenvironment and original cell-cell interactions of a patient's tumor. This intact microenvironment is crucial to assess the effect of anticancer drugs that target it and to maximize patient-specific predictability.

Therefore, in a third type of cell model, pieces of tissue explanted from a patient are used for the cell culture. For example, in oncology applications, the tissue may comprise explanted “intact” tumor tissues that preserve their native tumor microenvironment. Such patient-derived explanted tissue are also referred to herein as patient-derived explants (PDE) and it is believed that 3D cell culture of such PDEs can provide a more accurate physiological replication compared to 3D culture models that use spheroids or organoids. However, such PDEs typically have a low cell viability ex vivo (3 days in average) limiting their uses.

Developers have noted that the low cell viability of the PDEs may in part be due to the cell culture devices which are conventionally used with PDES, which are microfluidic devices (also known as lab-on-chips or organ-on-chips). Conventional microfluidic devices comprise a plurality of wells for housing the PDEs, and which wells are fed by cell culture fluid through channels. One or more pumps can control the fluid flow rate through the channels. The microfluidic devices are sealed other than a fluid inlet and a fluid outlet for the fluid supply and removal from the channels.

The microfluidic devices are typically made of polydimethylsyloxane (PDMS) which Developers have noted are susceptible to absorbing small hydrophobic molecules, such as the drug compounds being tested as well fluorescent markers of treatment response. Therefore, use of PDMS in a microfluidic device can result in inaccurate dosing of the drug compounds being tested, inaccurate dose-response interpretations, cross-contamination, and higher background fluorescence.

Furthermore, microfluidic devices made of PDMS are challenging and expensive to mass-produce using established techniques like injection molding and extrusion, which may limit the commercial usefulness of microfluidic platforms made fully of PDMS.

Finally, although PDEs are a preferred cell model, the size of PDE that is typically required for avoiding hypoxia (i.e. sub-milimeter) is not compatible with standard processes for transferring tissues into paraffin for histopathological evaluation as standard paraffin embedding processes are designed for macroscopic tissues.

In view of the above, Developers of the present technology have devised an improved cell culture device for 3D culture. In certain embodiments, the cell culture device provides an improvement over conventional cell culture devices as cell viability in the cell culture device during cell culture is increased.

From a broad aspect, the cell culture device comprises different components that can be attached together, including a component with recesses and/or channels for receiving the cell models and having a removable base. The removeable base may be made of a porous material permitting air diffusion therethrough for improved oxygen access to the cell models during the culture, thereby improving viability of the cultured cell models.

In accordance with a first aspect of the technology, there is provided a cell culture device comprising: a chip component having: a top face and a bottom face, at least one channel formed in the chip component, the at least one channel being open at the top face, and having a base spaced from the bottom face; at least one well portion extending from the base of the at least one channel to the bottom face of the chip component, the at least one well portion being open at the bottom face of the chip component and at the base of the at least one channel; and a membrane component configured to be positioned on the bottom face of the chip component to close the open well portion, the membrane component having at least one porous portion which is aligned with the open well portion when the membrane component and the chip component are assembled together, the at least one porous portion of the membrane component being permeable to gas and impermeable to liquid.

In some embodiments, the cell culture device further comprises a top cover component configured to be positioned on the top face of the chip component to substantially cover the open channel at the top face. The top cover may be configured to seal the open channel at the top face.

In some embodiments, the top cover component includes at least one opening that can fluidly communicate with the at least one channel when the top cover component is assembled with the chip component.

In some embodiments, the cell culture device further comprises a bottom cover component configured to be positioned beneath the membrane component, the bottom cover component having at least one opening, which when the at least one opening is aligned with the porous portion of the membrane component and the open well portion of the chip component, permits gaseous communication to the at least one channel through the bottom cover component.

In some embodiments, the at least one opening in the bottom cover component has a width which is wider than a width of the at least one channel in the chip component.

In some embodiments, the cell culture device has an assembled configuration in which the top cover component and the bottom cover component sandwich the chip component and the membrane component therebetween.

In some embodiments, the cell culture device further comprises a spacing member for spacing the membrane component from a support surface on which the cell culture device is to be supported.

In some embodiments, the spacing member is a retaining member for securing together the membrane component and the chip component, and which retaining member extends outwardly from the membrane component and away from the chip component. In other embodiments the spacing member is connected to the bottom cover.

In some embodiments, the retaining member comprises a bolt, nail or a screw extending through the membrane component and optionally the chip component.

In some embodiments, the cell culture device further comprises a retaining member for securing together one or more of the membrane component, the chip component, the top cover component and the bottom cover component.

In some embodiments, the retaining member comprises one or more of: nuts and bolts, magnetic components, clips and straps.

In some embodiments, the at least one channel comprises an inlet and an outlet.

In some embodiments, one or more of the chip component, the top cover component, the bottom cover component, and the membrane component are not made of poly dimethylsiloxane (PDMS).

In some embodiments, the membrane component has a surface which is functionalized.

In some embodiments, one or both of the top cover component and the bottom cover component is made from a rigid polymer.

In some embodiments, the top cover component is transparent or semi-transparent.

In some embodiments, one or both of the top face and the bottom face of the chip component is oxygen plasma treated.

In some embodiments, the membrane component is attached to the chip component.

In some embodiments, a width of the well portion is less than a width of the at least one channel.

In some embodiments, the at least one channel comprises a plurality of well portions spaced from one another along the at least one channel.

In some embodiments, the plurality of well portions are spaced from one another such that they can be received on a standard histology slide.

In some embodiments, the cell culture device further comprises a tissue model in the well portion when the chip component and the membrane component are assembled together.

In some embodiments, the cell culture device further comprises culture medium in the well portion when the chip component and the membrane component are assembled together.

In some embodiments, the at least one channel comprises a plurality of channels separated from one another.

From another aspect, there is provided a cell culture device comprising: a chip component having a top face, a bottom face, at least one channel formed in the chip component, the at least one channel being open at the top face, and having a base spaced from the bottom face; and at least one well portion extending from the base of the at least one channel to the bottom face of the chip component, the at least one well portion being open at the bottom face of the chip component and at the base of the at least one channel; and a membrane component configured to be positioned on the bottom face of the chip component to close the open well portion, the membrane component having at least one porous portion which is aligned with the open well portion when the membrane component and the chip component are assembled together; a top cover component configured to be positioned on the chip component to substantially cover the open channel at the top face of the chip component; a bottom cover component configured to be positioned beneath the membrane component, the bottom cover component having openings, wherein when the cell culture device is assembled, the top cover component and the bottom cover component sandwich the chip component and the membrane component therebetween.

In some embodiments, the bottom cover component has at least one opening, which when the at least one opening is aligned with the porous portion of the membrane component and the open well portion of the chip component, permits gaseous communication to the at least one channel through the bottom cover component.

In accordance with another broad aspect of the present technology, there is provided a cell culture device for cell culture comprising: a chip component comprising: a plate having a plate top face and a plate bottom face, and at least one channel formed in the plate, the at least one channel having side walls defined within the plate and extending between the plate top face and the plate bottom face, the channel having an open channel top face and an open channel bottom face; a top cover component configured to be positioned on the plate top face to substantially cover the open channel top face of the at least one channel; a membrane component configured to be positioned on the plate bottom face to close the open channel bottom face of the at least one channel, the membrane component being permeable to gas and impermeable to liquid; a bottom cover component configured to be positioned beneath the membrane component, the bottom cover component having openings for permitting gaseous communication to the at least one channel through the membrane component, wherein when the cell culture device is assembled, the top cover component and the bottom cover component sandwich the chip component and the membrane component.

In some embodiments, the openings in the bottom cover component have a width which is wider than a width of the at least one channel at the plate bottom face.

In some embodiments, the cell culture device further comprises clamping members for removably clamping together the top cover component and the bottom cover component.

In some embodiments, the clamping members comprise one or more of: nuts and bolts, magnetic components, and clips.

In some embodiments, the top cover component includes one or both of an inlet and an outlet for fluidly communication with the at least one channel of the chip component when assembled.

In some embodiments, one or more of the chip component, the top cover component, the bottom cover component, and the membrane component are not made of poly dimethylsiloxane (PDMS).

In some embodiments, the membrane component has a surface which is functionalized.

In some embodiments, one or both of the top cover component and the bottom cover component is made from a rigid polymer.

In some embodiments, the top cover component is transparent or semi-transparent.

In some embodiments, one or both of the top face and the bottom face of the chip component is oxygen plasma treated.

In some embodiments, the membrane component is attached to the chip component.

In some embodiments, the at least one channel includes a well portion, the well portion comprising a portion along the at least one channel in which a width of the at least one channel at the channel bottom face is narrower than a width of the at least one channel at the channel top face.

In some embodiments, the at least one channel comprises a plurality of well portions spaced from one another along the at least one channel.

In some embodiments, the components are assembled together and the well portion includes a sample of tissue positioned therein.

In some embodiments, the plurality of well portions are spaced from one another such that they can be received on a standard histology slide.

In some embodiments, the chip component, the top and bottom cover components and the membrane component are assembled together and further comprises culture medium in the at least one channel.

In some embodiments, the at least one channel comprises a plurality of channels separated from one another.

In accordance with a second broad aspect of the present technology, there is provided a cell culture device for cell culture comprising: a chip component comprising at least one channel having an open channel top face and an open channel bottom face; a top cover component configured to be positioned on the chip component to substantially cover the open channel top face of the at least one channel; a membrane component configured to be positioned on the chip component to cover the open channel bottom face of the at least one channel; a bottom cover component configured to be positioned beneath the membrane component, the bottom cover component having openings, wherein when the cell culture device is assembled, the top cover component and the bottom cover component sandwich the chip component and the membrane component therebetween.

In accordance with a third broad aspect of the present technology, there is provided a kit for a cell culture device, the kit comprising: at least one a chip component comprising at least one channel having an open channel top face and an open channel bottom face; at least one top cover component configured to be positioned on the chip component to cover the open channel top face of the at least one channel; at least one membrane component configured to be positioned on the chip component to close the open channel bottom face of the at least one channel; at least one bottom cover component configured to be positioned beneath the membrane component, the bottom cover component having openings.

In certain embodiments in which the removeable base is made of a porous material permitting air diffusion therethrough, the cell models have improved oxygen access during cell culture. As the cell models, in use, will rest on or near the base, a diffusion distance between the cell models and the ambient air is minimized.

Furthermore, the cell culture device may be provided as a kit of parts comprising different bases with different gaseous diffusion properties, and/or different chip components with different size/shape of recesses and/or channels. This can provide flexibility to a user of the cell culture device by permitting the user to build a cell culture device having desired properties appropriate to a given need, thereby allowing the user to control the cell culture conditions.

Advantageously, the modular configuration of the cell culture device permits ease of manufacture.

In certain embodiments, the modular configuration of the cell culture device provides versatility as the membrane component may be configured to have different properties to the chip component, such as gas permeability, and chemical functionalization.

Furthermore, the modular configuration of the cell culture device may also permit ease of post-culture processing of the cell model. For example, in certain embodiments, the cell culture device is configured such that the cell model can be embedded in paraffin for subsequent investigation by histopathology. The paraffin embedding of the cell model can be performed whilst the cell culture device is in the assembled configuration, and the paraffin embedded cell model can be removed for subsequent histopathology by removing the membrane component. This can enable an entire exposed face of the paraffin embedded cell model to be exposed to the histopathological solutions, thereby maximising the post-culture testing capability.

In certain embodiments, the recesses and/or channels are sized for microfluidic flow. Due to the small volume of the channels, a required volume of cell culture fluid is also decreased as well as drugs and metabolites added to the cell culture fluid.

Additionally, in certain non-limiting embodiments, the cell culture device may be optically transparent allowing for optical imaging to be done directly through its top surface without disturbing the samples trapped in the wells.

In the context of the present specification, unless provided expressly otherwise, the words “first”, “second”, “third”, etc. have been used as adjectives only for the purpose of allowing for distinction between the nouns that they modify from one another, and not for the purpose of describing any particular relationship between those nouns.

Implementations of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE FIGURES

Further features and advantages of the present technology will become apparent from the following detailed description, taken in combination with the appended drawings, in which:

FIG. 1 illustrates an assembled side view of a cell culture device for cell culture having a chip component, top and bottom cover components and a membrane component, in accordance with various non-limiting embodiments of the present disclosure;

FIG. 2 illustrates an exploded isometric view of the cell culture device of FIG. 1, in accordance with various non-limiting embodiments of the present disclosure;

FIG. 3 illustrates an exploded side view of the cell culture device of FIG. 1, in accordance with various non-limiting embodiments of the present disclosure;

FIG. 4 illustrates an isometric view of the cell culture device of FIG. 1 with the top and bottom cover components made transparent in order to see channels on the chip component more clearly, in accordance with various non-limiting embodiments of the present disclosure;

FIG. 5 illustrates an assembled top view of the cell culture device of FIG. 1 in which the top cover component is transparent, in accordance with various non-limiting embodiments of the present disclosure;

FIG. 6 illustrates a cross-sectional view of the cell culture device of FIG. 5 through the line A-A, in accordance with various non-limiting embodiments of the present disclosure;

FIG. 7 illustrates a zoomed-in view of the circled portion of the cell culture device of FIG. 6, in accordance with various non-limiting embodiments of the present disclosure;

FIG. 8 illustrates an isometric view of the cell culture device of FIG. 1 depicting modular inlet members, in accordance with various non-limiting embodiments of the present disclosure;

FIG. 9A illustrates an isometric view of the chip component of the cell culture device of FIG. 1, in accordance with various non-limiting embodiments of the present disclosure;

FIG. 9B illustrates a zoomed-in view of a portion of the chip component of FIG. 9A; and

FIG. 10 illustrates levels of oxygen in the cell culture device of FIG. 1 versus time (h) for different membrane component porosities, along with the biological Michaelis-Menten constant for oxygen (Km) and hypoxia threshold, in accordance with various non-limiting embodiments of the present disclosure.

It is to be understood that throughout the appended drawings and corresponding descriptions, like features are identified by like reference characters. Furthermore, it is also to be understood that the drawings and ensuing descriptions are intended for illustrative purposes only and that such technology do not provide a limitation on the scope of the claims.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future.

With these fundamentals in place, we will now consider some non-limiting examples to illustrate various implementations of aspects of the present technology.

In accordance with various non-limiting embodiments, there is provided a cell culture device which can be used for 3D cell culture of cell models. The cell culture device may be used to house the cell models whilst exposing them to various stimuli (e.g. drugs added to the culture fluid such as chemotherapy drugs or external treatments such as radiation applied to the cell culture device).

The cell models suitable for culture in the cell culture device are not particularly limited. In certain embodiments, the cell models comprise patient-derived explants (PDEs) such as, but not limited to, explanted tissue pieces. In certain embodiments, the PDEs comprises pieces of explanted tissue cut into disk-like samples having a diameter of about 380 micron and a thickness of about 300 micron. In other embodiments, the cell models comprise explanted tissue pieces which are roughly spherical with diameters between about 300 microns and about 700 microns. In yet other embodiments, the cell models comprise explanted tissue pieces which are about 1 mm wide.

Embodiments of the present cell culture device may also be used with isolated cells, or cells within a tissue matrix such as spheroids and organoids.

The cell culture device can be used to expose the cell models to various treatments and assess cell reaction thereto. Embodiments of the cell culture device can be used to assess treatment efficacy for a patient by exposing the patient's cells to the treatment in the cell culture device before the treatment is applied to the patient. Embodiments of the cell culture device provide a certain level of breathability to the cell models housed therein which may improve their viability whilst permitting liquid retention therein.

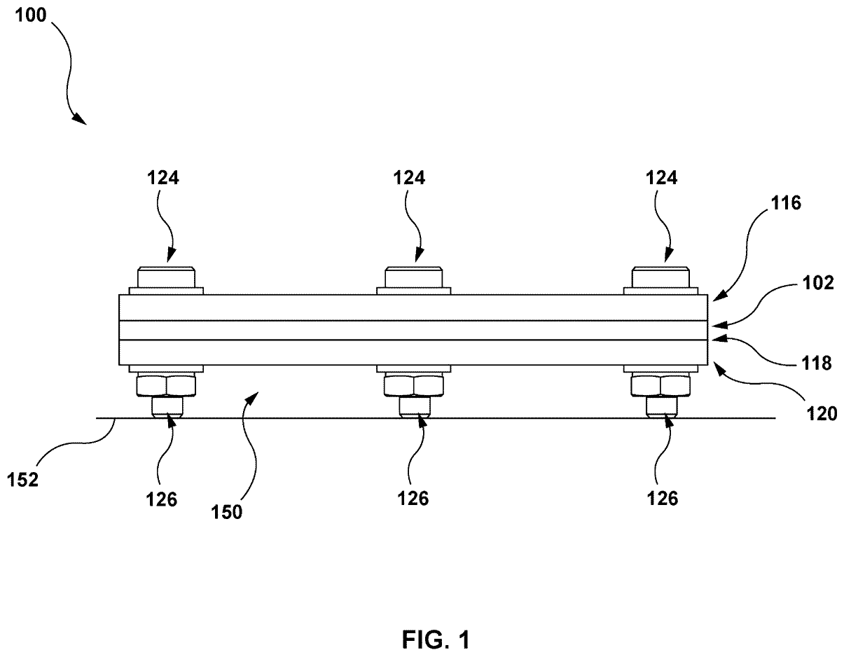

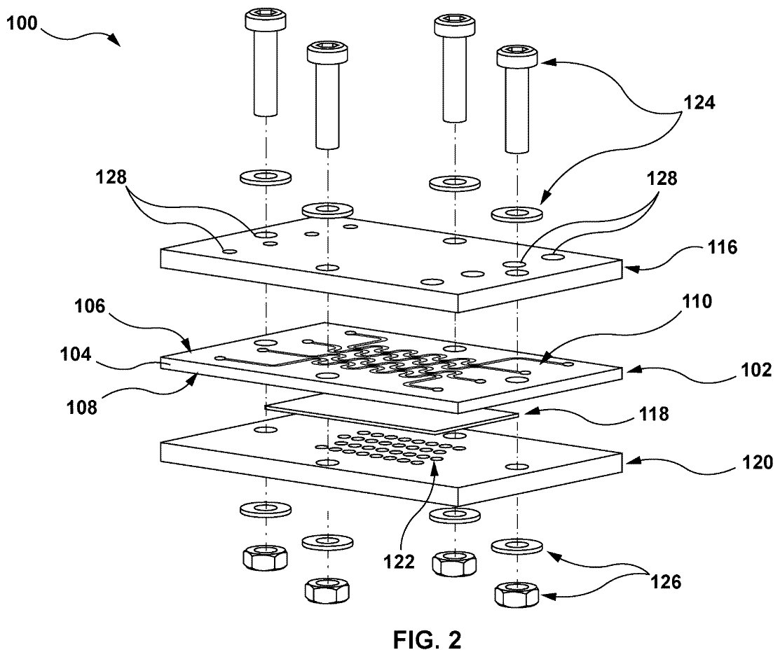

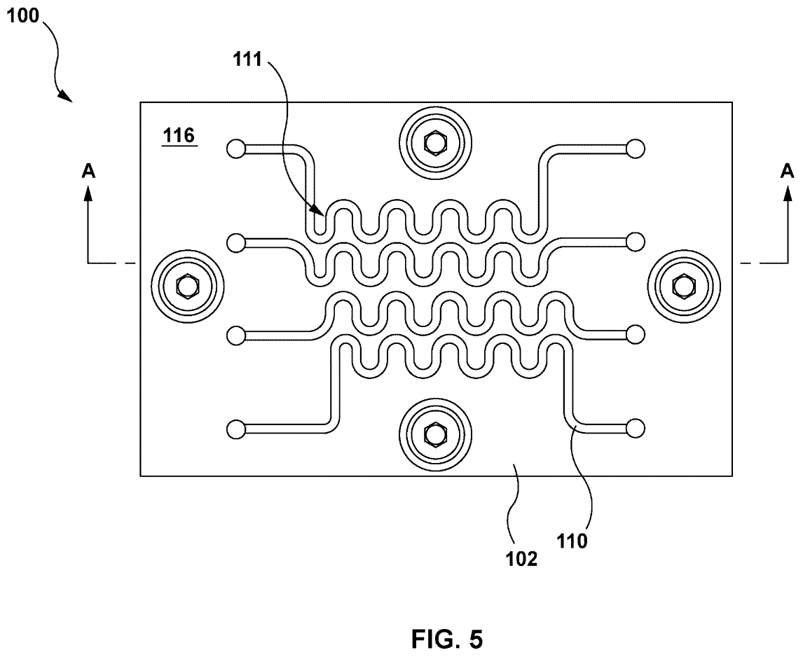

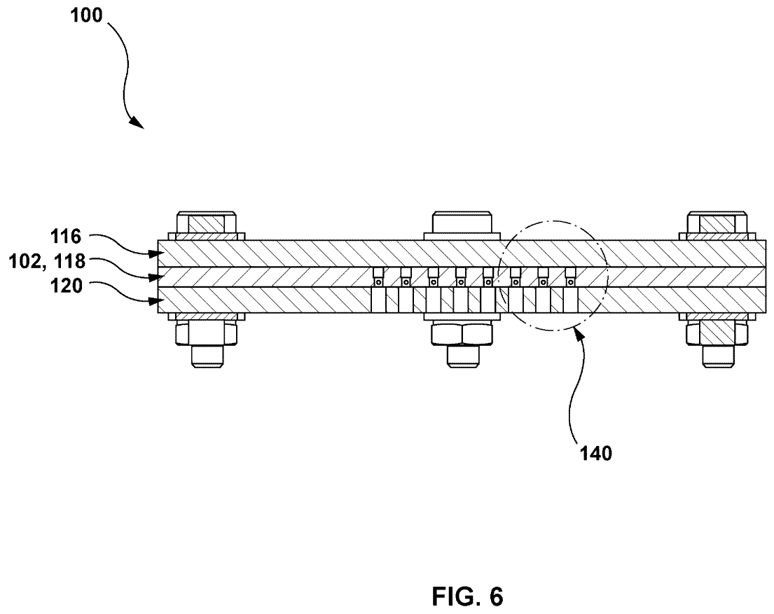

Referring to FIGS. 1-10, there is provided a cell culture device 100 which is modular and comprises components that can be laid one on top of each other and attached together in a sandwich-like layered configuration. The components comprise a chip component 102 with channels 110 formed therein. Each channel 110 has well portions 134 spaced apart from each other along the channel 110. Each well portion 134 is a recess within the chip component 102 extending from a base 111 of the channel 110 downwardly to a bottom face 108 of the chip component 102. Each well portion 134 is open at the bottom face 108 of the chip component 102, as well as at the base 111 of the channel 110. Each well portion 134 is configured to house the cell model 146 when the cell culture device 100 is assembled.

The cell culture device 100 also has a membrane component 118 configured to be positioned on the bottom face 108 of the chip component 102 and to cover the well portions 134 at the bottom face 108 of the chip component 102 when the chip component 102 and the membrane component 118 are assembled together. The cell culture device 100 also comprises top and bottom covers 116, 120 that sandwich the chip component 102 with the membrane component 118 when assembled. When the different components of the cell culture device 100 are assembled, the well portions 134 at the bottom face 108 of the chip component 102 are fluidly sealed enabling retention therein of the cell model and culture fluid. One or more retention members, such as the clamping members 124 and 126, may be provided to hold together the top and bottom cover components 116, 120 with the chip component 102 and the membrane component 118 sandwiched therebetween.

It is to be noted that the cell culture device 100 may include additional components not listed herein. In certain embodiments, the cell culture device 100 may omit one or more of the components listed above, such as the top cover component 116. The components will each be described in further detail below.

### Chip Component

The chip component 102 is plate-like and has a top face 106 and the bottom face 108. In certain embodiments, the top and bottom faces 106, 108 are parallel to one another.

There are four channels 110 formed in the chip component 102 of the illustrated embodiment. In other embodiments, there may be provided any number of channels 110, such as a single channel. The channels 110 may be implemented as those described in “Micro-dissected tumor tissues on chip: an ex vivo method for drug testing and personalized therapy”, Lab Chip, published on Dec. 2, 2015, the contents of which are hereby incorporated by reference.

Each channel 110 has side walls 112 and the base 111. The base extends generally parallel to the top and bottom faces 106, 108, and is spaced from both the top and bottom faces 106, 108. The side walls 112 extend generally transversely from the base 111 of each channel 110, although other relative configurations are within the scope of the present technology. For example, an angle of the side walls 112 may be more than or less than 90 degrees relative to the base 111. Each channel 110 is open at the top face 106 of the chip component 102, and closed at the base 111 other than the open well portions 134. In other words, the channels 110 of the chip component 102 are open-faced channels or slots and are generally closed at the base 111 other than recesses formed in the base 111 which are the well portions 134. As best seen in FIG. 1, in certain non-limiting embodiments, the chip component 102 is a microfluidic chip component meaning that the channels are sized and shaped for microfluidic flow. In other embodiments (not shown), the cell culture device 100 may have uses which are not microfluidic. Each channel 110 has an inlet 130 and an outlet 132 for fluid flow therethrough.

It is to be noted that the channels 110 as illustrated are distinct channels which are separated from each other. In other embodiments, the plurality of channels 110 may be fluidly connected instead of fluidly separate as herein illustrated. Each channel 110 has a sinusoidal configuration, although other shapes are also possible

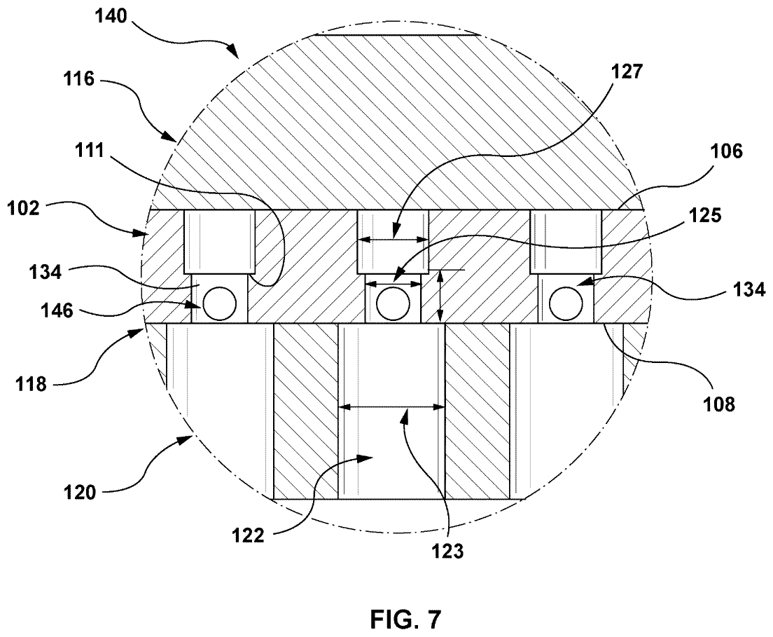

Turning now to the well portions 134 which are configured to trap the cell models 146 therein when the cell culture device 100 is assembled. In the embodiment illustrated, each channel 110 is provided with eight well portions 134, although in other embodiments, there may be more or less well portions 134 provided per channel 110. It will be appreciated that providing a plurality of well portions 134 per channel permits multiple sample testing with the same channel 110 conditions. In certain embodiments, each well portion 134 has a width 125 of about 600 microns and a height of about 500 microns. The width 125 of a given well portion 134 may be narrower than a width 127 of the given channel 110 in which it is formed (as shown in FIG. 7).

It is to be understood that different well portions 134 in the cell culture device 100 may have the same or different dimensions. The well portions 134 are illustrated as having a cuboid configuration but in other embodiments, the well portions 134 may have any other shape such as cylindrical, conical, etc.

The size and shape of each well portion 134 may be designed as a function of the size of the cell model 146 to be housed in the well portion 134. This is the case particularly when the cell models are micro-dissected tissues.

The dimensions of each well portion 134 may be selected so as to provide a desired trapping stability of the cell model 146, a desired cell viability of the cell model 146 within a desired culture time (e.g. no loss of cell proliferation activity and no significant cell death in a 15 day period), a desired shear stress generated when changing the culture medium and a desired metabolism of the cell models 146.

By way of example, the cell models 146 may have a diameter of between about 300 microns and about 700 microns. Accordingly, each well portion 134 may have size and shape sufficient to house the largest cell model 146 (assuming all the well portions 134 have the same size), for example a cross-sectional footprint of about 800 microns x about 800 microns, and a height of about 700 microns.

The given channel 110 must have a width sufficient to allow the largest cell model 146 to pass through to the well portion 134, for example a width of at least 800 microns.

In another example, the cell models 146 may have a diameter of up to about 1 mm. Accordingly, the cell culture device 100 has well portions 134 sized to house the cell models 146, and channels 110 of sufficient size to permit the cell models 146 to pass therethrough.

In certain non-limiting embodiments, the cell culture device 100, when assembled, at least one of the plurality of well portions 134 may include the cell model 146 positioned therein. Additionally, the cell culture device 100, when assembled, may comprise a culture medium in the well portion 134 or the channel 110.

The channels 110 may be implemented as described in “Micro-dissected tumor tissues on chip: an ex vivo method for drug testing and personalized therapy”, Lab Chip, Jan. 21, 2016, the contents of which are hereby incorporated by reference.

### Top Cover Component

The top cover component 116 is configured to be positioned on the top face 106 of the chip component 102 to substantially close the channels 110 at the top face 106. In certain non-limiting embodiments, the top cover component 116 may include openings 128 such that, when the cell culture device 100 is assembled, the openings 128 may fluidly communicate with respective channels 110 of the chip component 102. A respective opening 128 may align with a respective channel inlet 130 or a channel outlet 132 when the cell culture device 100 is assembled.

By substantially close is meant that the top cover component 116 may cover the channels 110 except for a portion of the channels that may remain uncovered because of the openings 128, and/or other openings defined therein for receiving the retaining members, such as the clamping members 124, 126. In other embodiments in which there are no openings 128 provided in the top cover component 116, the top cover component 116 may function to fluidly seal the channels 110 at the top face 106.

In certain non-limiting embodiments, the top cover component 116 may be transparent or semi-transparent allowing for optical imaging to be done directly through the top cover component 116 without disturbing the trapped cell models.

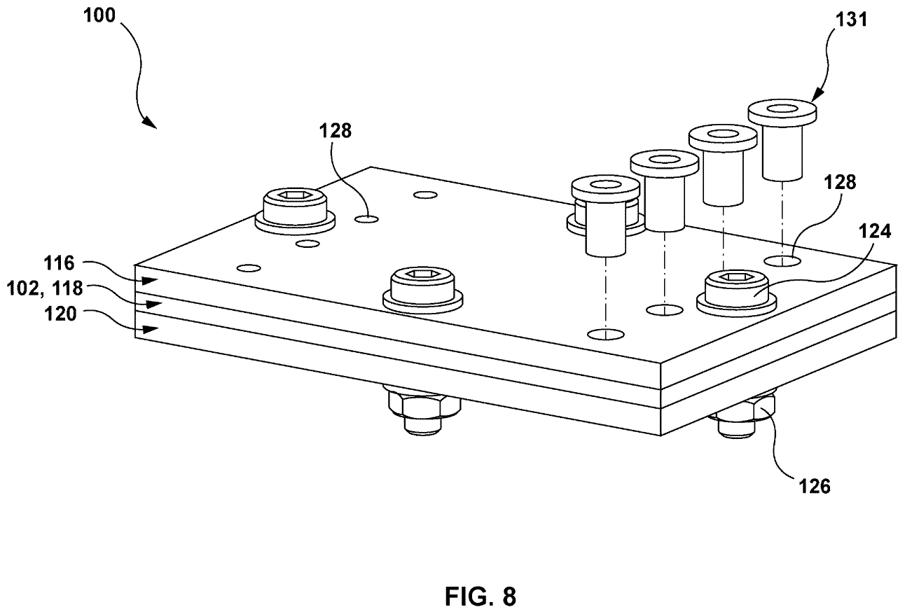

In certain non-limiting embodiments, the chip component 102 and/or the top cover component 116 may be configured such that the cell models 146 can be positioned into the channels 110 by injection. In this respect and as best seen in FIG. 8, an inlet member 131 may be provided for insertion into the opening 128 of the top cover 116. The cell models 146 and/or fluids may be injected into each channel 110 through the inlet member 131 and the inlet 130. In some embodiments, the outlet 132 may be used to remove the cell models 146 and/or the fluid. The inlet 130 may have a diameter of about 3 mm, and the outlet 132 may have a diameter of about 2 mm.

In certain embodiments, the top cover component 116 may include channels that line up with the channels 110 of the chip component 102. In yet other embodiments, further channels may be provided on one or both of the top cover component 116 and the bottom cover component 120 to thereby form one or more networks of channels throughout the cell culture device 100.

### Membrane Component

As mentioned above, the membrane component 118 is configured to be positioned on the bottom face 108 of the chip component 102 to close the bottom face 108 at the well portions 134. The membrane component 118 thus functions as a base of the well portions 134 when the cell culture device 100 is assembled.

In certain embodiments, the membrane component 118 may be made of a different material or have a different configuration than the chip component 120. For example, in certain embodiments, the membrane component 118 may be permeable to gas, such as air, and impermeable to liquids thereby permitting gaseous diffusion to the well portions 134 whilst retaining liquid within the well portions 134 and the channels 110. In this respect, at least a portion of the membrane component 118 may be porous. In certain embodiments, the membrane component 118 may be porous throughout. In other embodiments, the membrane component 118 may be porous only in areas that line up with the well portions 134 of the chip component 102 when the cell culture device 100 is assembled. The membrane component 118 is sized and shaped to cover all the well portions 134. In this respect, the membrane component 118 may be smaller than the chip component 102 as long as the well portions 134 are covered by it. In certain embodiments, the membrane component 118 is the same size and shape as the chip component 102.

In certain non-limiting embodiments, a porosity of the membrane component 118 may be selected depending on the intended application for which the cell culture device 100 is to be used. The membrane component 118 with specific attributes may be selected and assembled in the cell culture device 100. By way of example, the membrane component 118 with a relatively lower porosity may be selected to create hypoxic culture conditions for cell models 146, and a membrane component 118 with a relatively higher porosity may be selected to create hyperoxic culture conditions for cell models 146.

Some non-limiting examples of the material of the membrane component 118 may include one or more of polytetrafluoroethylene (PTFE) polymer, expanded-PTFE (e-PTFE) polymer, polyethylene-PTFE (Pe-PTFE) polymer, Fluorinated ethylene propylene (FEP) polymer, Polyvinylidene fluoride (PVDF) polymer, Polydimethylsiloxane (PDMS) polymer, Polyethylene terephthalate (PET) polymer, Polylactic acid (PLA) polymer, polymethylpentene (PMP) polymer, poly(1-trimethylsilyl-1-propyne) (PTMSP) polymer, polymethylated polymer such as polymethylated poly(diphenylacetylene), and/or any other suitable polymer that may achieve sufficient gas permeability through any suitable mechanism.

In certain non-limiting embodiments, the membrane component 118 may have hydrophobic properties for minimizing or preventing fluid leakage out of the bottom of the cell culture device 100 through the well portions 134 when the cell culture device 100 is assembled.

In certain non-limiting embodiments, the membrane component 118 may be attached to the chip component 102, such as by one or more of: adhesive, heat lamination.

In certain other non-limiting embodiments, the membrane component 118 may be separate from the chip component 102 and connectable thereto, such as by clamping between the top and bottom cover components 116, 120.

In certain non-liming embodiments, the membrane component 118 may be functionalized with functional groups such as one or more of: proteins, biomolecules or a chemically defined component having CFx, NHx, Si, OH, CO2 groups, without being limited thereto.

In certain non-limiting embodiments, the membrane component 118 may have controlled pore sizes, from no porosity to very high porosity, to control gas exchanges across it. In certain embodiments, a pore size of the membrane component 118 may be selected according to a desired gas exchange. In certain embodiments, the membrane component 118 may have pores with different pore sizes.

In certain non-liming embodiments, the membrane component 118 may have sensors to detect specific analytics within the cell culture device 100.

### Bottom Cover Component

The bottom cover component 120 is configured to be positioned adjacent the membrane component 118. In certain non-limiting embodiments, the bottom cover component 120 has openings 122 for permitting gaseous communication to the well portions 134 through the membrane component 118. The bottom cover component 120 is sized and shaped to cover the membrane component 118 and optionally the chip component 102. The openings 122 are aligned with the well portions 134 when the cell culture device 100 is assembled. In certain embodiments, the bottom cover component 120 is the same size and shape as one or both of the chip component 102 and the membrane component 118.

In certain non-limiting embodiments, a given opening 122 in the bottom cover component 120 may have a width 123 which is wider than the width 125 of a given well portion. In certain non-limiting embodiments, the width 123 of the openings 122 in the bottom cover component 120 may be wider than the width 127 of the channel 110. Having the width 123 of the openings 122 wider than the width 125 of the given well portion 134 and/or the width 127 of the channel 110, may provide an improved flow of oxygen to the cell model 146 in the well portion 134.

The openings 122 may have any appropriate cross-sectional shape, such as circular, square, etc. A thickness of the bottom cover component 120 is also not limited, and any suitable thickness may be selected to achieve the desired gas diffusion.

In certain non-limiting embodiments, one or more of the chip component 102, the top cover component 116 and the bottom cover component 120 may be made from a rigid polymer. Some non-limiting examples of materials that can be used to form one or more of the chip component 102, and the top and/or bottom cover components 116, 120 include polycarbonates (PC), polystyrenes (PS), polyethylene (PE), cyclic olefin co-polymers (COG), cyclic olefin polymers (COP).

It is to be noted that one or more of the chip component 102, the top cover component 116, the membrane component 118, and the bottom cover component 120 may be made of a material which is not PDMS. The advantage of one or more of the top cover component 116, the membrane component 118, and the bottom cover component 120 not being made of PDMS is that any detrimental effect by PDMS is avoided. In other words, it may be advantageous to avoid or minimize components that will contact the cell models 146 or the cell culture fluid being made of PDMS.

### Retention Members

As mentioned above, when the cell culture device 100 is assembled, the top cover component 116 and the bottom cover component 120 sandwich the chip component 102 and the membrane component 118 therebetween. Retention members may be provided to retain the cell culture device 100 in the assembled state. In certain embodiments, the retention members are removeable and permit disassembly of the cell culture device 100. In the illustrated embodiment, the retention members comprise the clamping members 124 and 126 which are nuts and bolts. Corresponding openings are provided in each of the components of the cell culture device 100 through which the bolt can be threaded.

In other embodiments, in addition to or instead of the clamping members 124 and 126, any suitable retention members may be provided, for example, magnets, clips, bands, straps, or the like.

In certain non-limiting embodiments, the retention members, such as the clamping members 124, 126 may have one or more of the following functions: i) to keep all the components of the cell culture device 100 together with sufficient pressure to avoid leaking of the fluids, ii) to be removable in order to permit the user to remove and replace components of the device during an experiment, and iii) to ensure proper alignment of the components.

FIG. 7 illustrates a cross-sectional view through the cell culture device 100 including the channels 110 and the well portions 134, as well as the associated interfaces at the top and bottom faces 106, 108 of the chip component 102. More particularly, as illustrated in FIG. 7, in certain non-limiting embodiments, an interface between the top cover component 116 and the chip component 102 when the cell culture device 100 is assembled comprises a fluid seal therebetween. An interface between the bottom face 108 of the chip component 102 and the membrane component 118 when the cell culture device 100 is assembled may also include a fluid seal. Alternatively, or in addition, the bottom cover component 120 and the membrane component 118 may also comprise a fluid seal.

In certain non-limiting embodiments, the fluid seal is achieved through a clamping pressure from the retaining members. In certain non-limiting embodiments, the fluid seal may be obtained or enhanced by an additional sealing means such as one or more of adhesive, plasma treatment (e.g., oxygen plasma treatment), and/or with sufficient surface pressure (e.g., o-ring surface seal) or any other suitable sealing agent. In certain non-limiting embodiments, one or both of the top face 106 and the bottom face 108 of the chip component 102 may be oxygen plasma treated.

In certain non-limiting embodiments, the top cover component 116, the chip component 102, the membrane component 118 and the bottom cover component 120 may be sealed in a manner permitting disassembly of the individual components by the user as required.

It is to be noted that control of oxygen available to the cell models 146, according to embodiments of the present technology, allows for control of hypoxia within the cell models 146. As such, a precise control of the oxygen is useful for simulating certain tumor microenvironment conditions.

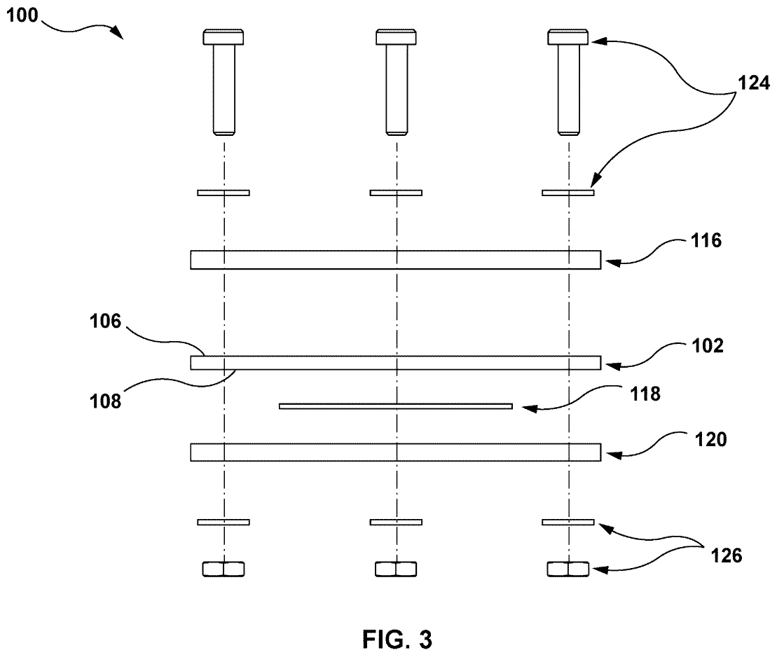

In certain non-limiting embodiments, the availability of oxygen to the cell models 146 can be controlled by the membrane component 118 and the openings 122 of the bottom cover component 120 (as shown in FIGS. 3 and 7). Selection of appropriate gaseous diffusion properties of the membrane component 118 and/or the size and positioning of the openings 122 can modulate the oxygen availability to the cell models. Airflow can thus be provided from the bottom cover component 120 to the membrane component 118 for ensuring that the cell models 146 have sufficient oxygen access.

Thus, it may be said that certain embodiments of the cell culture device 100, may provide improved viability of the cultured cell models 146 from the point of view of sufficient oxygen availability. The cell culture device 100 may be configured to provide sufficient oxygen to the cell models 146 despite the use of gas-impermeable thermoplastic material for the chip component 102, and despite the static culture of the cell models 146 (i.e., without continuous perfusion and replenishment of the culture medium in the channels).

More particularly, the chip component 102 may minimize a distance between the cultured cell models 146 and ambient air, thus providing sufficient oxygen for cell viability. In certain non-limiting embodiments, there may be a gap 150 between the bottom cover component 120 and a support surface 152 on which the bottom cover component 120 is resting. In doing so, air may pass through the gap 150 below the bottom cover component 120, then through the openings 122 in the bottom cover component 120 and may reach the plurality of well portions 134 though porous regions of the membrane component 118. By flowing through the bottom of the plurality of well portions 134, oxygen may diffuse through the culture medium before getting to the cell models 146, as the cell models 146 may be positioned near to the membrane component 118.

The cell models 146 may also need to be provided with sufficient nutrients, e.g., glucose, that can be provided with the culture medium in which the cell models 146 may be submerged. For cell models 146 which are tissue samples to avoid hypoxia in its core and obtain improved viability, a replenishment of the nutrient may be required, and a critical size of the cell model 146 may be considered.

In order to preserve greater viability of the cell models 146 than standard explant culture, in addition to the providing sufficient oxygen to the cell models 146, the cell culture device 100 may provide a mechanism that allows the replenishment of nutrients.

In certain non-limiting embodiments, the cell culture device 100 may allow for sufficient nutrient access by having a volume of culture medium big enough to provide the amount of nutrients required by the cell models 146 over a period of, for example, 24 hours. To replenish the nutrients the user may only have to change the culture medium every day or two simplifying the process compared to conventional microfluidic platforms requiring a setup of pumps to ensure the perfusion of the medium culture.

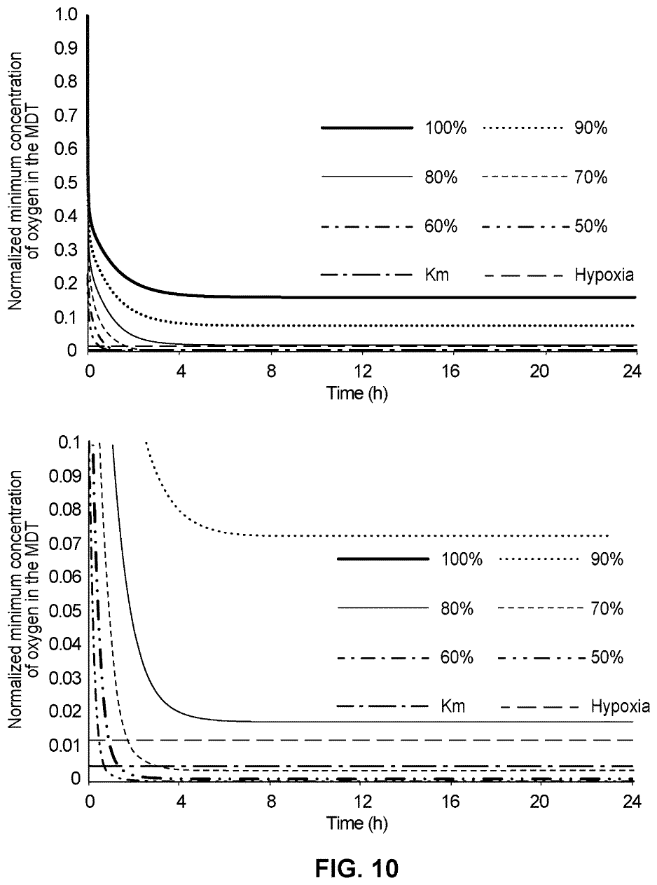

FIG. 10 illustrates levels of oxygen in an embodiment of the cell culture device 100 versus time (h) for different porosities of the membrane component 118, along with the biological Michaelis-Menten constant for oxygen (km) and hypoxia threshold, in accordance with various non-limiting embodiments of the present disclosure. The cell model 146 used were cut pieces of explant tissue having a size of about 380 micron diameter and 300 micron height. More particularly, FIG. 10 illustrates a normalized minimum concentration of oxygen in the cell model 146 versus time for different porosities of the membrane component 118 demonstrating that i) oxygen levels are above the threshold for viability of the cell model 146 in the cell culture device 100 and ii) that oxygen levels in the cell model 146 are a function of the porosity of the membrane component 118. The Michaelis-Menten constant is used to indicate the threshold under which oxygen concentration limits uptake kinetics.

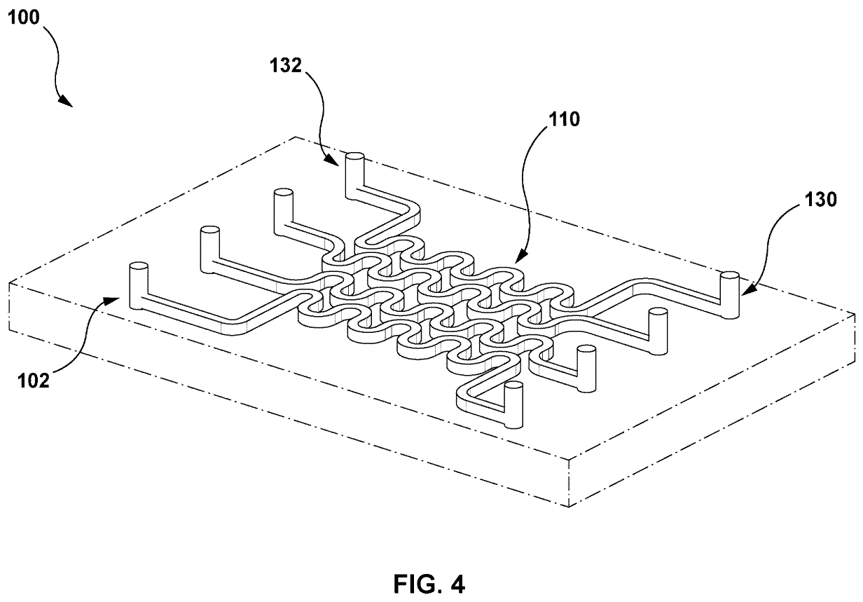

In addition to previously discussed benefits, the cell culture device 100 may provide different methods for loading the cell models 146 in the cell culture device 100. In one non-limiting embodiment, referring to FIGS. 4 and 8, when the cell models 146 are cut pieces of explant tissue having a size of about 380 micron diameter and 300 micron height, the cell models 146 may be injected with a fluid at the inlet 130 of the assembled cell culture device 100 with a pipette (not illustrated). By controlling the flow of the fluid with the pipette, the user may stop when the cell models 146 are over a given one of the plurality of well portions 134 and let the cell models 146 sediment.

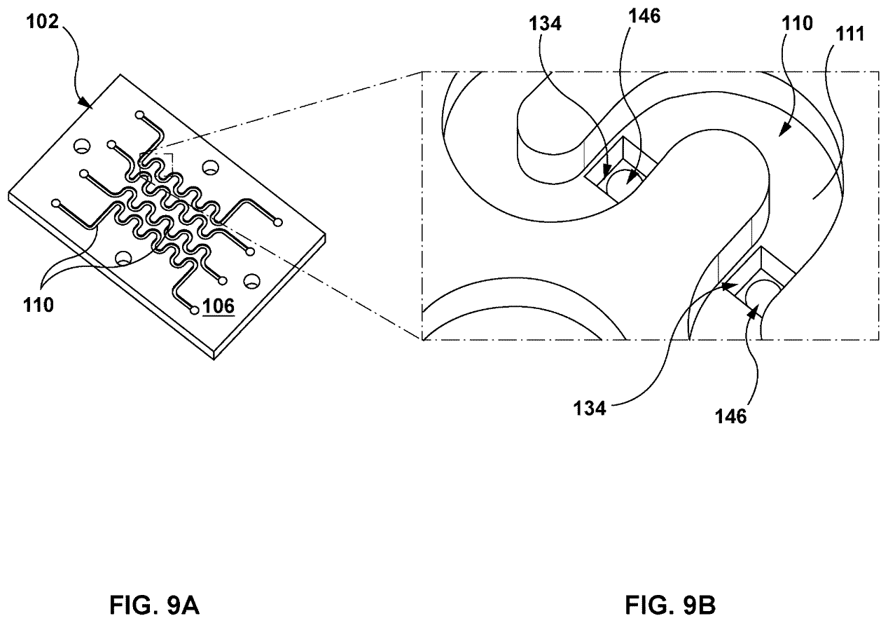

In another non-limiting embodiment, the cell models 146 may be placed directly in the plurality of well portions 134 while the membrane component 118 and the bottom cover component 120 are in place and with the top cover component 116 is removed (as shown in FIG. 9).

Furthermore, the cell culture device 100 may be provided as a kit with different types of any of the components such as different versions of: the membrane component 118 (e.g. having different gaseous diffusion properties), the top cover component 116 (e.g. having different sizes of openings 128), the bottom cover component 120 (e.g. having different size openings), and the chip component 102 (e.g. having different size channels 110 and/or well portions 134) that would enable different applications. By way of example, with same chip component 104 and the bottom cover component 120, a first type of the top cover component 116 may be used to inject a specific volume of culture medium in the well portions 134 only, then a second type of the top cover component 116 may be positioned in place to close the channel 110 and let the user perform the culture medium changes. Finally, a third type of the top cover component 116 may be used for the transfer of cell models 146 into paraffin for embedding.

In certain non-limiting embodiments, the plurality of well portions 134 may be designed to fit in the standard dimensions of a histology cassette. Such arrangement may enable the cell culture device 100 to be compatible with the Paraffin-Embedded Lithography process. The cell culture device 100 thus facilitates the generation of histopathology endpoints, reduces the fixation time of the cell models 146 and the transfer of the cell models 146 into paraffin.

In this respect, embodiments of the cell culture device 100 may be compatible with methods, such as those described in U.S. patent application Ser. No. US 2020/0124507, “Medium-embedded samples,” filed on Oct. 19, 2018, the contents of which are hereby incorporated by reference.

Broadly, the method comprises separating the top cover component 116 of the cell culture device 100 from the chip component 102, placing embedding medium in the channels 110 including the well portions 134 such that the embedding medium immerses the tissue models 146 in the well portions 134; allowing the embedding medium to form a block of embedding medium including the tissue models 146 embedded therein; and separating the chip component 102 from the block of embedding medium to obtain the medium-embedded-sample-block.

Placing the embedding medium in the well portions 134 may comprise filling the well portions 134 with the embedding medium, filling a given channel 110 above the well portions 134 with the embedding medium, and forming a layer of embedding medium above the well portions 134. The embedding medium may be a paraffin-based composition. In certain embodiments, before placing the embedding medium in the well portions 134, the method may comprise dehydrating the tissue models 146. The embedding medium may be in a liquid state when immersing the tissue models 146, and the allowing the embedding medium to form the block of embedding medium comprises allowing a change in a state of the embedding medium from the liquid state to a solid state. Placing the embedding medium in the well portions 134 may comprise immersing the chip component 102 in the embedding medium. In certain embodiments, this comprises placing the chip component 102 and the membrane component 118 (and optionally the top and bottom covers 116, 120) within an open container, the open container being deeper than a height of the chip component 102, and adding the embedding medium to fill the open container over the chip component 102 such that the chip component 102 is immersed in the embedding medium.

Once the embedding medium with the tissue models 146 embedded therein has solidified (also referred to as “first block”), the first block is separated from the components of the cell culture device 100. The first block can then be further processed to allow compatibility with standard histopathology techniques. In certain embodiments, the first block is further treated by placing the first block in a mold, optionally with more paraffin, and re-melting the first block and allowing the tissue models 146 to settle at the base of the metal mold before re-solidifying. This further step can achieve a flat surface of paraffin with the aligned tissue models 146 therein which can facilitate the thin section processing.

In other embodiments, the chip component may be flushed with other materials for preservation such as OCT (cryopreservation), agarose, hydrogels, polyacrylamide, and collagen.

In certain non-limiting embodiments, the cell culture device 100 may be fabricated with different fabrication techniques due to the different parts that compose the cell culture device 100. By way of example, the top cover component 116 and the bottom cover component 120 may consist of simple rigid plastic parts with holes and microchannels depending on the embodiment. The top cover component 116 and the bottom cover component 120 may be made by laser cutting or micro milling depending on the thermoplastic used (3 mm thick polycarbonate for instance may not be compatible with laser cutting and may be micromilled with a CNC). These techniques allow rapid prototyping and are convenient for medium volume of production. For a larger production volume ≥10 000, the top cover component 116 and the bottom cover component 120 may be produced by injection molding, as the thermoplastics used may be injectable or may be hot embossed.

In certain non-limiting embodiments, the chip component 102 may be fabricated by soft lithography or soft thermoplastic lithography depending on the material to be used. In certain non-limiting embodiments, hot embossing may also be employed.

In certain non-limiting embodiments, the membrane component 118 may be cut into dimensions that cover the plurality of well portions 134. In certain non-limiting embodiments, the membrane component 118 may be placed under the chip component 102. In certain non-limiting embodiments, the membrane component 118 may be laminated to the bottom cover component 120 in order to reduce the number of parts the user may be required to assemble. In certain non-limiting embodiments, the lamination may be done by spraying a biocompatible adhesive to the bottom face 108 of the chip component 102 and adding the membrane component 118 to the bottom face 108.

Once all the components of the cell culture device 100 have been fabricated, the cell culture device 100 may be assembled. In certain non-limiting embodiments, the cell culture device 100 may be performed by the end user. In so doing, a manufacturing cost of the cell culture device 100 may be reduced and it may also enable the user to use different variants of the top cover component 116 and the bottom cover component 120 or the membrane components 118 based on the application and use. The assembly of the cell culture device 100 may include putting the components in a sandwich formation with the following order from top to bottom: the top cover component 116, the chip component 102, the membrane component 118 and the bottom cover component 120. It is to be noted that while the components of the cell culture device 100 may be assembled without any alignment effort as the components may be designed to fit together when assembled. In certain non-limiting embodiments, the top face 106 may be treated with oxygen plasma to increase adherence and sealing with the top cover component 116.

Without limiting the scope of present disclosure, prior to culturing the cell models 146 within the cell culture device 100, the cell culture device 100 may be prepared for culturing. To this end, the plurality of channels 110 may be treated with a plutonic F108 solution for few hours (e.g., 24 hours). Then the plutonic F108 solution may be washed out and the cell models 146 may be inserted into the cell culture device 100 with a culture medium.

In certain non-limiting embodiments, the cell culture device 100 may be used for drug screening in the context of drug discovery by enabling the direct testing of therapeutic agents, or radiation treatments, on cell models 146 cultured in the cell culture device 100. Many assays may be designed with the cell culture device 100 and the users may control the type of treatment, treatment regimen, types of endpoints, time of endpoints, etc.

It is to be noted that the cell culture device 100 may assist in performing various measurements. By way of example, the cell culture device 100 may be imaged as the top cover component 116 may be optically clear, culture medium may be collected after culture to be analyzed, the cell models 146 may also be collected from the cell culture device 100 and then be characterized in any suitable manner such as flow cytometry or RNA sequencing, or the like.

In certain non-limiting embodiments, the cell culture device 100 may be used in clinical settings for the personalization of the treatment of a patient with cancer. In this embodiment, the cell models may comprise MDTs derived from a patient tumor biopsy which is loaded into to the cell culture device 100 (in the well portions) and then may be exposed to a candidate treatment to assess the patient-specific sensibility to the treatment. This assay may provide predictive information regarding the patient-specific response to the treatment. This assay may provide, to a medical doctor, valuable insight to better design the patient's treatment plan. This assay may also be executed before any treatment is given to a patient and may save the patient from undergoing an ineffective cancer treatment and from undesirable side effects if the ex vivo assay identifies the patient as non-responder of the treatment. For this application, similarly, the patient derived MDTs may be tested ex vivo in the cell culture device 100 and may undergo a direct transfer to paraffin for subsequent histological analysis. This predictive assay may serve as a companion diagnostic tool, along with an approved treatment, to help patients and doctors prescribe the right personalized treatment.

In addition to benefits of the cell culture device 100 previously discussed, the cell culture device 100 may consume minimal amount of reagent due to the low volume channels 110 and thus reduces cost of experiments compared to macroscopic vessels. Additionally, the cell culture device 100 may preserve cell model ex vivo viability and proliferation activity 5× times longer (almost 15 days) than the conventional techniques, e.g., tissue slice model and Farcast' tumoroid.

The cell culture device 100 may increase throughput of assays using primary cell models (such as MDTs) by 10× times compared to other conventional techniques, e.g., tissue slice model.

In the cell culture device 100, spheroids and PDEs may not require extracellular matrix substitutes like matrigel™ for the 3D growth of the cells. This offers a cost-effective means for cultivating human-relevant cell models.

Also, the dimensions of the chip component 102 may be compatible with clinical radiotherapy and small animal radiotherapy machines.

Some of the additional benefits of embodiments of the cell culture device 100 may include one or more of: i) capability to grow cells in a 3D architecture, ii) circumvent the need to buy specialized equipment related to microfluidics, iii) capability to do low to high throughput experiments, iv) accelerate and ease the fixation and transfer into paraffin of the PDEs or spheroids, v) reduce the quantity of reagent needed per experiment (compared to multi well plates), vi) a means to refine and reduce animal use for research, vii) reduce absorption of small hydrophobic molecules in the microfluidic system (compared with PDMS-based systems), viii) a more physiologically-relevant models (than 2D culture) to test drugs in vitro and evaluate their effect, ix) a cost-effective mean to evaluate drug effect on microtissues with their native tumor microenvironement (when PDEs of cancer tissue are used with the cell culture device 100), x) a cost-effective mean to identify sensitive or resistant in vivo models with a 3D in vitro model, xi) provide crucial predictive information for the personalization of a patient's treatment, xii) complement the genetic information used to tailor a treatment, xiii) reduce the probability to give an ineffective treatment to a patient (saving time and pain for the patient and cost for healthcare system), and xiv) reduce the quantity of reagent needed per experiment (compared to multi well plates).

In certain non-limiting embodiments, the components of the cell culture device 100 may be provided as a kit. The kit may include at least one chip component 102. The kit may further include at least one top cover component 116 configured to be positioned on the chip component 102. The kit may further include at least one membrane component 118 configured to be positioned on the chip component 102 to close the well portions 134 at the bottom face 108 of the chip component 102. The kit may further include at least one bottom cover component 120 configured to be positioned beneath the membrane component 118, the bottom cover component 120 having the openings 122.

It is to be noted that in addition to the components of the cell culture device 100, the kit may include instructions for use of the kits such as for assembling the components of the cell culture device 100. Without limiting the scope of present disclosure, the instructions may be printed on a packaging of the kit or may be printed on a paper and the paper is included in the kit.

It will also be understood that, although the embodiments presented herein have been described with reference to specific features and structures, it is clear that various modifications and combinations may be made without departing from such technologies. The specification and drawings are, accordingly, to be regarded simply as an illustration of the discussed implementations or embodiments and their principles as defined by the appended claims, and are contemplated to cover any and all modifications, variations, combinations or equivalents that fall within the scope of the present technology.

1. A cell culture device comprising:
a chip component having:
a top face and a bottom face,
at least one channel formed in the chip component, the at least one channel being open at the top face, and having a base spaced from the bottom face;
at least one well portion extending from the base of the at least one channel to the bottom face of the chip component, the at least one well portion being open at the bottom face of the chip component and at the base of the at least one channel; and
a membrane component configured to be positioned on the bottom face of the chip component to close the at least one open well portion, the membrane component having at least one porous portion which is aligned with the at least one open well portion when the membrane component and the chip component are assembled together, the at least one porous portion of the membrane component being permeable to gas and impermeable to liquid.

2. The cell culture device of claim 1, further comprising:
a top cover component configured to be positioned on the top face of the chip component to substantially cover the channel at the top face.

3. The cell culture device of claim 2, wherein the top cover component includes at least one opening that can fluidly communicate with the at least one channel when the top cover component is assembled with the chip component.

4. The cell culture device of claim 1, further comprising:
a bottom cover component configured to be positioned beneath the membrane component, the bottom cover component having at least one opening, which when the at least one opening is aligned with the porous portion of the membrane component and the at least one open well portion of the chip component, permits gaseous communication to the at least one channel through the bottom cover component.

5. The cell culture device of claim 4, wherein the at least one opening in the bottom cover component has a width which is wider than a width of the at least one channel in the chip component.

6. The cell culture device of claim 2, wherein the cell culture device has an assembled configuration in which the top cover component and the bottom cover component respectively sandwich the chip component and the membrane component therebetween.

7. The cell culture device of claim 1, further comprising a spacing member for spacing the membrane component from a support surface on which the cell culture device is to be supported.

8-11. (canceled)

12. The cell culture device of claim 1, wherein the at least one channel comprises an inlet and an outlet.

13. The cell culture device of claim 1, wherein one or more of the chip component, the top cover component, the bottom cover component, and the membrane component are not made of polydimethylsiloxane.

14. The cell culture device of claim 1, wherein the membrane component has a surface which is functionalized.

15. (canceled)

16. The cell culture device of claim 1, wherein the top cover component is transparent or semi-transparent.

17. The cell culture device of claim 1, wherein one or both of the top face and the bottom face of the chip component is oxygen plasma treated.

18. The cell culture device of claim 1, wherein the membrane component is attached to the chip component.

19. The cell culture device of claim 1, wherein a width of the at least one well portion is less than a width of the at least one channel.

20. The cell culture device of claim 1, wherein the at least one channel comprises a plurality of well portions spaced from one another along the at least one channel.

21. The cell culture device of claim 20, wherein the plurality of well portions are spaced from one another such that they can be received on a standard histology slide.

22. The cell culture device of claim 1, further comprising a tissue model and culture medium in the at least one well portion when the chip component and the membrane component are assembled together.

23. (canceled)

24. The cell culture device of claim 1, wherein the at least one channel comprises a plurality of channels separated from one another.

25. A cell culture device comprising:
a chip component having a top face, a bottom face, at least one channel formed in the chip component, the at least one channel being open at the top face, and having a base spaced from the bottom face; and at least one well portion extending from the base of the at least one channel to the bottom face of the chip component, the at least one well portion being open at the bottom face of the chip component and at the base of the at least one channel; and
a membrane component configured to be positioned on the bottom face of the chip component to close the open well portion, the membrane component having at least one porous portion which is aligned with the open well portion when the membrane component and the chip component are assembled together;
a top cover component configured to be positioned on the chip component to substantially cover the open channel at the top face of the chip component;
a bottom cover component configured to be positioned beneath the membrane component, the bottom cover component having openings,
wherein when the cell culture device is assembled, the top cover component and the bottom cover component sandwich the chip component and the membrane component therebetween.

26. The cell culture device of claim 25, wherein the bottom cover component has at least one opening, which when the at least one opening is aligned with the porous portion of the membrane component and the open well portion of the chip component, permits gaseous communication to the at least one channel through the bottom cover component.
